(19) United States
(12) Patent Application Publication
(10) Pub. No.: US 2024/0390470 A1
(43) Pub. Date: Nov. 28, 2024

(54) Non-Toxin Antigens For Clostridioides Difficile Vaccine

(71) Applicants: Vanderbilt University, Nashville, TN (US); The United States Government As Represented by the Department of Veterans Affairs, Washington, DC (US)

(72) Inventors: D. Borden LACY, Nashville, TN (US); Audrey THOMAS, Nashville, TN (US)

(21) Appl. No.: 18/671,007

(22) Filed: May 22, 2024

Related U.S. Application Data
(60) Provisional application No. 63/503,576, filed on May 22, 2023.

(51) Int. Cl. A61K 39/08; A61K 31/4164; A61K 38/14; A61K 39/00; A61P 31/04

(52) U.S. Cl. CPC A61K 39/08; A61K 31/4164; A61K 38/14; A61P 31/04; A61K 2039/55544

(57) ABSTRACT

Disclosed herein are C. difficile surface proteins that can serve as antigens in a vaccine to prevent C. difficile infection. While many trials have been focused on the use of the C. difficile toxins as antigens, the neutralization of toxins does not prevent colonization by the organism.

### CROSS-REFERENCE TO RELATED APPLICATIONS

This claims benefit of U.S. Provisional Application No. 63/503,576, filed May 22, 2023, which is hereby incorporated herein by reference in its entirety.

### STATEMENT OF GOVERNMENT INTEREST

This invention was made with Government Support under Grant No. AI174999 awarded by the National Institutes of Health. The Government has certain rights in the invention.

### BACKGROUND OF THE INVENTION

Clostridioides difficile infection (CDI) is caused by two potent exotoxins in the colon, engenders mild-to-severe diarrhea, and can cause life-threatening pseudomembranous colitis, toxic megacolon, and colonic perforation. Upwards of $4.8 billion is spent treating CDI, 500,000 are infected, and 29,000 people die of CDI each year in the United States. Previous vaccines against CDI have been unsuccessful, likely due to targeting toxins systemically, rather than the bacterial cell surface at the source of infection to prevent colonization.

### SUMMARY OF THE INVENTION

Disclosed herein are C. difficile surface proteins that can serve as antigens in a vaccine to prevent C. difficile infection. While many trials have been focused on the use of the C. difficile toxins as antigens, the neutralization of toxins does not prevent colonization by the organism.

An in silico study identified highly-conserved C. difficile proteins as candidate vaccine antigens. Therefore, disclosed herein is a vaccine composition comprising 1, 2, 3, 4, 5, 6, 7, 8, 9, 10, 11, 12, 13, 14, 15, or 16 C. difficile proteins (or immunogenic fragments thereof) selected from the group consisting of a polysaccharide deacetylase (WP_003436667), Glucosaminidase domain-cont. protein (WP__009890365), N-acetylmuramoyl-L-alanine amidase (WP_009893723), C40 peptidase 1 (WP_009890599), C40 peptidase 2 (WP_009892230), FIgK (flagellar hook-associated protein) (WP_009892584), FlgD (flagellar basal body mod. protein) (WP_009888079), FIgG (flagellar basal body rod) (WP_009888104), FIgE (flagellar hook-basal body protein) (WP_009888081), DUF3794 domain-containing protein (WP 009890363), S8 peptidase (WP_009890437), N-acetylglucosaminidase (WP 009892971), FliD (flagellar cap protein) (WP 009888060), NlpC/P60-family protein (WP 009889145), Cell wall protein 12 (WP_009893729), and Cell wall protein 21 (WP_009893883). In some embodiments, the vaccine composition comprises a combination of FIgG, FIgE, and FIgK proteins in a ternary complex. In some embodiments, the vaccine composition comprises a combination of one or more of the 16 C. difficile proteins (or immunogenic fragments thereof) with one or more of the C. difficile toxins (TcdA (WP_343099982.1), TcdB (WP_009895693.1), CDT (AY341253.1 and AF271719); or immunogenic fragments thereof).

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 shows FIgG and FIgK exhibit substantial patient serum IgG reactivity when tested by ELISA.

FIG. 2 shows FIgG, FIgE, and FIgK are predicted to interact and sit outside of the C. difficile peptidoglycan.

FIG. 3 shows the flagellar basal body rod protein FIgG is predicted to natively oligomerize into at least an 11-mer in C. difficile.

FIG. 4 shows negative-stain electron microscopy of FIgG suggests native oligomerization amongst heterogeneity.

FIG. 5 shows representative electron microscopic views of oligomeric FIgG.

FIG. 6 shows negative-stain electron microscopy of mixed FIgG+E+K highlights heterogenous, flexible complexes and aggregates.

FIG. 7 contains representative electron microscopic views of the putative FIgG+E+K complex.

FIG. 8 is a schematic of prime-boost vaccination with non-toxin antigens and subsequent C. difficile challenge model in mice.

FIG. 9 is a schematic of vaccination scheme to determine non-toxin antigen(s) protect against morbidity and moribundity, reduce colonization, and elicit an anti-NTA humoral response. Group 3 involves rectal instillation of FIgGEK+C40 peptidase 2; +/−dmLT.

FIGS. 10A to 10D show rectal instillation of FIgGEK reduces weight loss in both sexes.

FIGS. 11A and 11B show rectal instillation of FIgGEK reduces death in both sexes.

FIGS. 12A and 12B show rectal instillation of FIgGEK does not increase anti-FIgGEK serum IgG nor fecal IgA.

FIGS. 13A to 13D show rectal instillation of C40 pep 2 reduces weight loss in both sexes.

FIGS. 14A and 14B show rectal instillation of C40 pep 2 reduces death in both sexes.

FIGS. 15A and 15B show rectal instillation of C40 pep 2 does not increase anti-C40 pep 2 serum IgG nor fecal IgA.

FIG. 16 is a schematic of vaccination scheme to determine non-toxin antigen(s) protect against morbidity and moribundity, reduce colonization, and elicit an anti-NTA humoral response. Group 4 involves rectal instillation of polysaccharide deacetylase+dmLT only.

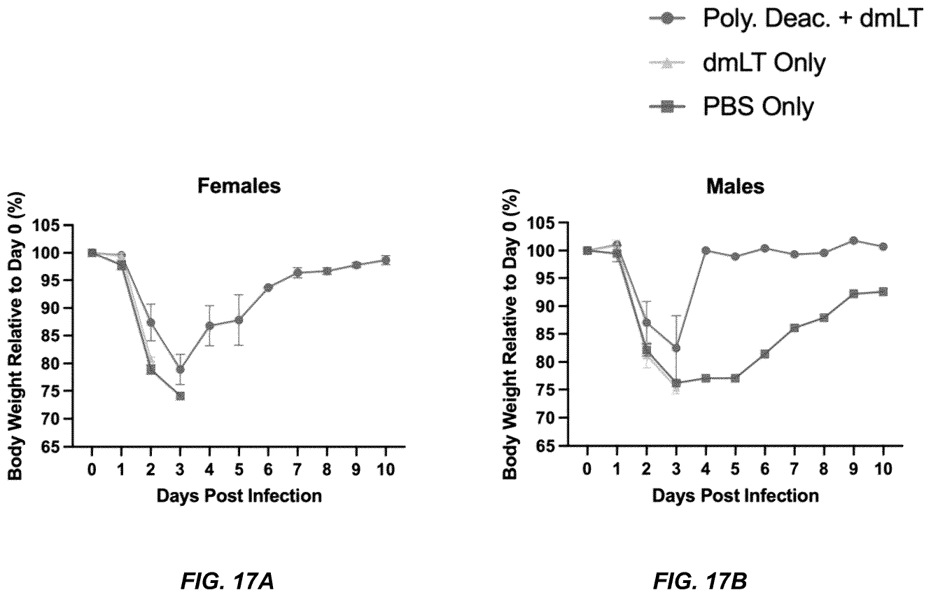

FIGS. 17A to 17D show rectal instillation of polysaccharide deacetylase reduces weight loss at 2 d.p.i.

FIGS. 18A and 18B show rectal instillation of polysaccharide deacetylase prevents some death.

FIGS. 19A and 19B show rectal instillation of polysaccharide deacetylase does not increase anti-polysaccharide deacetylase serum IgG nor fecal IgA.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half-life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

As used herein, the terms “Clostridioides difficile associated disease”, “Clostridioides difficile related disease”, “Clostridioides difficile-associated disease”, “Clostridioides difficile toxin-mediated disease”, “Clostridioides difficile infection”, and “CDAD” refer to diseases caused, directly or indirectly, by infection with Clostridioides difficile. In the older literature, the organism was named Clostridium difficile.

“Antigen” refers to a substance that induces a specific immune response when presented to immune cells of an organism. For example, an antigen may be a nucleic acid, a protein, a polypeptide, a peptide, a glycoprotein, a carbohydrate, a lipid, a glycolipid, a lipoprotein, a fusion protein, a phospholipid, or a conjugate of a combination thereof. An antigen may comprise a single immunogenic epitope, or a multiplicity of immunogenic epitopes recognized by a B-cell receptor (i.e., antibody on the membrane of the B cell) or a T-cell receptor. Antigen may be provided as a virus-like-particle (VLP) or a whole microbe or microorganism such as, for example, a bacterium or virion. The antigen may be an inactivated or attenuated live virus. The antigen may be obtained from an extract or lysate, either from whole cells or membrane alone; or antigen may be chemically synthesized or produced by recombinant means. An antigen may be administered by itself or with an adjuvant. A single antigen molecule may have both antigen and adjuvant properties.

### Vaccine Composition

The present invention also provides compositions comprising the disclosed non-toxic antigens. The compositions may be pharmaceutical compositions comprising the disclosed non-toxic antigens and a pharmaceutically acceptable carrier. The compositions used in the methods of the invention generally comprise, by way of example and not limitation, and effective amount of the disclosed non-toxic antigens (e.g., an amount sufficient to induce an immune response) or an antibody against the disclosed non-toxic antigens (e.g., an amount of a neutralizing antibody sufficient to mitigate infection, alleviate a symptom of infection and/or prevent infection). The pharmaceutical composition may further comprise pharmaceutically acceptable carriers, excipients, or stabilizers known in the art (see generally Remington, (2005) The Science and Practice of Pharmacy, Lippincott, Williams and Wilkins).

The disclosed non-toxic antigens may be used for methods for immunizing or treating humans and/or animals with the CDAD. Therefore, the disclosed non-toxic antigens may be used within a pharmaceutical composition. The pharmaceutical composition of the present invention may further encompass pharmaceutically acceptable carriers and/or excipients. The pharmaceutically acceptable carriers and/or excipients useful in this invention are conventional and may include buffers, stabilizers, diluents, preservatives, and solubilizers. Remington's Pharmaceutical Sciences, by E. W. Martin, Mack Publishing Co., Easton, Pa., 15th Edition (1975), describes compositions and formulations suitable for pharmaceutical delivery of the polypeptides herein disclosed. In general, the nature of the carrier or excipients will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e. g. powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In one embodiment the pharmaceutical composition may further comprise an immunostimulatory substance, such as an adjuvant. The adjuvant can be selected based on the method of administration and may include mineral oil-based adjuvants such as Freund's complete and incomplete adjuvant, Montanide incomplete Seppic adjuvant such as ISA, oil in water emulsion adjuvants such as the Ribi adjuvant system, syntax adjuvant formulation containing muramyl dipeptide, aluminum hydroxide or aluminum salt adjuvant (alum), polycationic polymer, especially polycationic peptide, especially polyarginine or a peptide containing at least two LysLeuLys motifs, immunostimulatory oligodeoxynucleotide (ODN) containing non-methylated cytosine-guanine dinucleotides (CpG) in a defined base context (e.g. as described in WO 96/02555) or ODNs based on inosine and cytidine (e.g. as described in WO 01/93903), or deoxynucleic acid containing deoxy-inosine and/or deoxyuridine residues (as described in WO 01/93905 and WO 02/095027), especially Oligo (dldC) 13 (as described in WO 01/93903 and WO 01/93905), neuroactive compound, especially human growth hormone (described in WO 01/24822), or combinations thereof. Such combinations are according to the ones e.g. described in WO 01/93905, WO 02/32451, WO 01/54720, WO 01/93903, WO 02/13857, WO 02/095027 and WO 03/047602. Preferably, the adjuvant is a double mutant of the heat labile enterotoxin, dmLT, as described in WO 1999047167A1.

Acceptable carriers, excipients, or stabilizers are nontoxic to recipients at the dosages and concentrations that are administered. Carriers, excipients or stabilizers may further comprise buffers. Examples of excipients include, but are not limited to, carbohydrates (such as monosaccharide and disaccharide), sugars (such as sucrose, mannitol, and sorbitol), phosphate, citrate, antioxidants (such as ascorbic acid and methionine), preservatives (such as phenol, butanol, benzanol; alkyl parabens, catechol, octadecyldimethylbenzyl ammonium chloride, hexamethoniuni chloride, resorcinol, cyclohexanol, 3-pentanol, benzalkonium chloride, benzethonium chloride, and m-cresol), low molecular weight polypeptides, proteins (such as serum albumin or immunoglobulins), hydrophilic polymers amino acids, chelating agents (such as EDTA), salt-forming counter-ions, metal complexes (such as Zn-protein complexes), and non-ionic surfactants (such as TWEEN™ and polyethylene glycol).

The pharmaceutical composition of the present invention may further comprise additional agents that serve to enhance and/or complement the desired effect. By way of example, to enhance the immunogenicity the disclosed non-toxic antigens being administered as a subunit vaccine, the pharmaceutical composition may further comprise an adjuvant.

An example of a pharmaceutical composition may be an immunogenic composition. The present invention provides immunogenic compositions comprising the disclosed non-toxic antigens. The immunogenic composition may further include a pharmaceutically acceptable carrier or other carriers and/or excipients in a formulation suitable for injection in a mammal. An immunogenic composition is any composition of material that elicits an immune response in a mammalian host when the immunogenic composition is injected or otherwise introduced. The immune response may be humoral, cellular, or both. A booster effect refers to an increased immune response to an immunogenic composition upon subsequent exposure of the mammalian host to the same immunogenic composition. A humoral response results in the production of antibodies by the mammalian host upon exposure to the immunogenic composition.

The immunogenic compositions of the present invention elicit an immune response in a mammalian host, including humans and other animals. The immune response may be either a cellular dependent response or an antibody dependent response or both; and further the response may provide immunological memory or a booster effect or both in the mammalian host. These immunogenic compositions are useful as vaccines and may provide a protective response by the mammalian subject or host to infection by strains of C. difficile.

The present invention also provides formulations comprising the disclosed non-toxic antigens for treating and preventing CDAD. In one embodiment, the formulation may include the disclosed non-toxic antigens, an adjuvant, and a pharmaceutically acceptable carrier. In another embodiment, the formulation the disclosed non-toxic antigens, or consists essentially of one or more of the disclosed non-toxic antigens. The formulation may comprise the disclosed non-toxic antigens and an adjuvant. The formulation may further include an additional antigen or a drug.

The formulation comprising the disclosed non-toxic antigens may be in liquid or dry form. A dry formulation may be easily stored and transported. Dry formulations break the cold chain required from the vaccine's place of manufacture to the locale where vaccination occurs. Alternatively, the dry, active ingredient of the formulation per se may be an improvement by providing a solid particulate form that is taken up and processed by antigen presenting cells. These possible mechanisms are discussed not to limit the scope of the invention or its equivalents, but to provide insight into the operation of the invention and to guide the use of this formulation in immunization and vaccination.

Dry formulations of the disclosed non-toxic antigens may be provided in various forms: for example, fine or granulated powders, lyophilized powder, uniform films, pellets, and tablets. It may be air dried, dried with elevated temperature, lyophilized, freeze or spray dried, coated or sprayed on a solid substrate and then dried, dusted on a solid substrate, quickly frozen and then slowly dried under vacuum, or combinations thereof. If different molecules are active ingredients of the formulation, they may be mixed in solution and then dried, or mixed in dry form only.

Formulations comprising the disclosed non-toxic antigens in liquid or solid form, such as a dry form, may be applied with one or more adjuvants at the same or separate sites or simultaneously or in frequent, repeated applications. The formulation may include other antigens such that administration of the formulation induces an immune response to multiple antigens. In such a case, the other antigens may have different chemical structures so as to induce an immune response specific for different antigens. At least one antigen and/or adjuvant may be maintained in dry form prior to administration. Subsequent release of liquid from a reservoir or entry of liquid into a reservoir containing the dry ingredient of the formulation will at least partially dissolve that ingredient.

Solids (e.g., particles of nanometer or micrometer dimensions) may also be incorporated in the formulation. Solid forms (e.g., nanoparticles or microparticles) may aid in dispersion or solubilization of active ingredients; provide a point of attachment for adjuvant, the disclosed non-toxic antigens, or both to a substrate that can be opsonized by antigen presenting cells, or combinations thereof. Prolonged release of the formulation from a porous solid formed as a sheet, rod, or bead acts as a depot.

At least one ingredient or component of the formulation (i.e., the disclosed non-toxic antigens, adjuvant, or drug) may be provided in dry form prior to administration of the formulation. This formulation may also be used in conjunction with conventional enteral, mucosal, or parenteral immunization techniques.

The formulation comprising the disclosed non-toxic antigens may be manufactured under aseptic conditions acceptable to appropriate regulatory agencies (e.g., Food and Drug Administration, EMEA for biologicals and vaccines. Optionally, components such as desiccants, excipients, stabilizers, humectants, preservatives, or combinations thereof may be included in the formulation even though they are immunologically inactive. They may, however, have other desirable properties or characteristics.

Processes for manufacturing a pharmaceutical formulation are well known. The components of the formulation may be combined with a pharmaceutically-acceptable carrier or vehicle, as well as any combination of optional additives (e.g., diluents, binders, excipients, stabilizers, desiccants, preservatives, colorings). The use of solid carriers, and the addition of excipients to assist in solubilization of dry components or stabilizers of immunogenic or adjuvant activity, are preferred embodiments. See, generally, Ullmann's Encyclopedia of Industrial Chemistry, 6th Ed. (electronic edition, 2003); Remington's Pharmaceutical Sciences, 22^{nd} (Gennaro, 2005, Mack Publishing); Pharmaceutical Dosage Forms, 2^{nd} Ed. (various editors, 1989-1998, Marcel Dekker); and Pharmaceutical Dosage Forms and Drug Delivery Systems (Ansel et al., 2005, Williams & Wilkins).

Good manufacturing practices are known in the pharmaceutical industry and regulated by government agencies (e.g., Food and Drug Administration, EMEA. Sterile liquid formulations may be prepared by dissolving an intended component of the formulation in a sufficient amount of an appropriate solvent, followed by sterilization by filtration to remove contaminating microbes. Generally, dispersions are prepared by incorporating the various sterilized components of the formulation into a sterile vehicle which contains the basic dispersion medium. For production of solid forms that are required to be sterile, vacuum drying or freeze drying can be used.

In general, solid dosage forms (e.g., powders, granules, pellets, tablets) can be made from at least one active ingredient or component of the formulation.

Suitable tableting procedures are known. The formulation may also be produced by encapsulating solid forms of at least one active ingredient, or keeping them separate from liquids in compartments or chambers. The size of each dose and the interval of dosing to the subject may be used to determine a suitable size and shape of the tablet, capsule, compartment, or chamber.

Formulations will contain an effective amount of the active ingredients (e.g., drug, antigen and adjuvant) together with carrier or suitable amounts of vehicle in order to provide pharmaceutically-acceptable compositions suitable for administration to a human or animal.

The relative amounts of active ingredients, such as amounts of the disclosed non-toxic antigens within a dose and the dosing schedule may be adjusted appropriately for efficacious administration to a subject (e.g., animal or human). This adjustment may also depend on the subject's particular disease or condition, and whether treatment or prophylaxis is intended. To simplify administration of the formulation to the subject, each unit dose contains the active ingredients in predetermined amounts for a single round of immunization.

There are numerous causes of polypeptide instability or degradation, including hydrolysis and denaturation. In the case of denaturation, the conformation or three-dimensional structure of the protein is disturbed and the protein unfolds from its usual globular structure. Rather than refolding to its natural conformation, hydrophobic interaction may cause clumping of molecules together (i.e., aggregation) or refolding to an unnatural conformation. Either of these results may entail diminution or loss of immunogenic or adjuvant activity. Stabilizers may be added to lessen or prevent such problems.

The formulation, or any intermediate in its production, may be pretreated with protective agents (i.e., cryoprotectants and dry stabilizers) and then subjected to cooling rates and final temperatures that minimize ice crystal formation. By proper selection of cryoprotective agents and use of pre-selected drying parameters, almost any formulation might be cryoprepared for a suitable desired end use.

It should be understood in the following discussion of optional additives like excipients, stabilizers, desiccants, and preservatives are described by their function. Thus, a particular chemical may act as some combination of recipient, stabilizer, desiccant, and/or preservative. Such chemical would be immunologically-inactive because it does not directly induce an immune response, but it increases the response by enhancing immunological activity of the antigen or adjuvant: for example, by reducing modification of the antigen or adjuvant, or denaturation during drying and dissolving cycles.

Stabilizers include cyclodextrin and variants thereof (see U.S. Pat. No. 5,730,969). Suitable preservatives such as sucrose, mannitol, sorbitol, trehalose, dextran, and glycerin can also be added to stabilize the final formulation (Howell and Miller, 1983). A stabilizer selected from nonionic surfactants, D-glucose, D-galactose, D-xylose, D-glucuronic acid, salts of D-glucuronic acid, trehalose, dextrans, hydroxyethyl starches, and mixtures thereof may be added to the formulation. Addition of an alkali metal salt or magnesium chloride may stabilize the disclosed non-toxic antigens, optionally including serum albumin and freeze-drying to further enhance stability. The disclosed non-toxic antigens may also be stabilized by contacting it with a saccharide selected from the group consisting of dextran, chondroitin sulfuric acid, starch, glycogen, insulin, dextrin, and alginic acid salt. Other sugars that can be added include monosaccharides, disaccharides, sugar alcohols, and mixtures thereof (e.g., glucose, mannose, galactose, fructose, sucrose, maltose, lactose, mannitol, xylitol). Polyols may stabilize a polypeptide, and are water-miscible or water-soluble. Suitable polyols may be polyhydroxy alcohols, monosaccharides and disaccharides including mannitol, glycerol, ethylene glycol, propylene glycol, trimethyl glycol, vinyl pyrrolidone, glucose, fructose, arabinose, mannose, maltose, sucrose, and polymers thereof. Various excipients may also stabilize polypeptides, including serum albumin, amino acids, heparin, fatty acids and phospholipids, surfactants, metals, polyols, reducing agents, metal chelating agents, polyvinyl pyrrolidone, hydrolyzed gelatin, and ammonium sulfate.

As an example, the disclosed non-toxic antigens can be stabilized in sucrose, trehalose, poly(lactic acid) (PLA) and poly(lactide-co-glycolide) (PLGA) microspheres by suitable choice of excipient or stabilizer (Sanchez et al., 1999). Sucrose, or trehalose may be advantageously used as an additive because it is a non-reducing saccharide, and therefore does not cause aminocarbonyl reactions with substances bearing amino groups such as proteins. Sucrose or trehalose may be combined with other stabilizers such as saccharides.

Additionally, the formulation comprising the disclosed non-toxic antigens may include therapeutic agents, such as e.g. anesthetics, analgesics, anti-inflammatoires, steroids, antibiotics, antiarthritics, anorectics, antihistamines, and antineoplastics. Examples of such therapeutic agents include lidocaine and nonsteroidal anti-inflammatory drugs (NSAID). In another embodiment, the therapeutic agents are antigens and adjuvants. In still another embodiment, the formulation comprising antigen and/or adjuvant may be applied separately but along with other therapeutic agents, such e.g anesthetics, analgesics, anti-inflammatories, steroids, antibiotics, antiarthritics, anorectics, antihistamines, and antineoplastics. In a preferred embodiment, the antibiotics are fidaxomicin, metronidazole or vancomycin.

Polymers may be added to the formulation and may act as an excipient, stabilizer, and/or preservative of an active ingredient as well as reducing the concentration of the active ingredient that saturates a solution used to dissolve the dry form of the active ingredient. Such reduction occurs because the polymer reduces the effective volume of the solution by filling the “empty” space. Thus, quantities of antigen/adjuvant can be conserved without reducing the amount of saturated solution. An important thermodynamic consideration is that an active ingredient in the saturated solution will be “driven” into regions of lower concentration. In solution, polymers can also stabilize and/or preserve the antigen/adjuvant-activity of solubilized ingredients of the formulation. Such polymers include ethylene or propylene glycol, vinyl pyrrolidone, and 0-cyclodextrin polymers and copolymers.

A single or unit dose of the formulation comprising the disclosed non-toxic antigens suitable for administration is provided. The amount of adjuvant and/or the disclosed non-toxic antigens in the unit dose may be anywhere in a broad range from about 0.001 μg to about 10 mg. This range may be from about 0.1 μg to about 1 mg; a narrower range is from about 5 μg to about 500 μg. Other suitable ranges are between about 20 μg to about 200 μg, such as e.g. about 20 μg, about 75 μg or about 200 μg.

The disclosed non-toxic antigens may be used as an antigen and may be presented to immune cells, and an antigen-specific immune response is induced. This may occur before, during, or after infection by a pathogen, such as C. difficile. Only the disclosed non-toxic antigens may be required, but no additional adjuvant, if the immunogenicity of the formulation is sufficient to not require adjuvant activity. The formulation may include an additional antigen such that application of the formulation induces an immune response against multiple antigens (i.e., multivalent).

Antigen-specific lymphocytes may participate in the immune response and, in the case of participation by B lymphocytes, antigen-specific antibodies may be part of the immune response. The formulations described above may include desiccants, excipients, humectants, stabilizers, and preservatives known in the art.

The formulation comprising the disclosed non-toxic antigens may be used to treat a subject (e.g., a human or animal in need of treatment such as prevention of disease, protection from effects of infection, reducing or alleviating the symptoms of a disease, such as CDAD, or combinations thereof). E.g. the formulation comprising the disclosed non-toxic antigens may be used to treat a subject at risk of CDAD, such as e.g. a subject with the following profile: i) a subject with a weaker immune system such as e.g. an elderly subject (e.g. a subject above 65 years of age) or a subject below 2 years of age; ii) an immunocompromised subject such as e.g. a subject with AIDS; iii) a subject taking or planning to take immunosuppressing drugs; iv) a subject with planned hospitalization or a subject that is in hospital; v) a subject in or expected to go to an intensive care unit (ICU); vi) a subject that is undergoing or is planning to undergo gastrointestinal surgery; vii) a subject that is in or planning to go to a long-term care such as a nursing home; viii) a subject with co-morbidities requiring frequent and/or prolonged antibiotic use; ix) a subject that is a subject with two or more of the above mentioned profiles, such as e.g. an elderly subject that is planning to undergo a gastrointestinal surgery; x) a subject with inflammatory bowel disease; and/or xi) a subject with recurrent CDAD such as e.g. a subject having experienced one or more episodes of CDAD.

The treatment may vaccinate the subject against infection by the pathogen or against its pathogenic effects such as those caused by toxin secretion. The formulation may be used therapeutically to treat existing disease, protectively to prevent disease, to reduce the severity and/or duration of disease, to ameliorate symptoms of disease, or combinations thereof.

The formulations comprising the disclosed non-toxic antigens may be delivered by various routes of administration including but not limited to intrarectal, oral, subcutaneous, intradermal, intravenous, intra-arterial, intramuscular, intracardial, intraspinal, intrathoracical, intraperitoneal, intraventricular, and/or sublingual routes.

The formulation may also comprise one or more adjuvants or combinations of adjuvants. Usually, the adjuvant and the formulation are mixed prior to presentation of the antigen but, alternatively, they may be separately presented within a short interval of time.

An adjuvant may be chosen to preferentially induce antibody or cellular effectors, specific antibody isotypes (e.g., IgM, IgD, IgA1, IgA2, secretory IgA, IgE, IgG1, IgG2, IgG3, and/or IgG4), or specific T-cell subsets (e.g., CTL, Th1, Th2 and/or TDTH) (see, for example, Munoz et al., 1990; Glenn et al., 1995).

Unmethylated CpG dinucleotides or motifs are known to activate B cells and macrophages (Stacey et al., 1996). Other forms of DNA can be used as adjuvants. Bacterial DNAs are among a class of structures which have patterns allowing the immune system to recognize their pathogenic origins to stimulate the innate immune response leading to adaptive immune responses (Medzhitov and Janeway, 1997, Curr. Opin. Immunol. 9 (1): 4-9). These structures are called pathogen-associated molecular patterns (PAMPs) and include lipopolysaccharides, teichoic acids, unmethylated CpG motifs, double-stranded RNA, and mannins. PAMPs induce endogenous signals that can mediate the inflammatory response, act as co-stimulators of T-cell function and control the effector function. The ability of PAMPs to induce these responses play a role in their potential as adjuvants and their targets are APCs such as macrophages and dendritic cells. PAMPs could also be used in conjunction with other adjuvants to induce different co-stimulatory molecules and control different effector functions to guide the immune response, for example from a Th2 to a Th1 response.

Other aspects of the invention is directed toward use of the disclosed non-toxic antigens as vaccinating agent. The vaccines or immunogenic compositions of the present invention may employ an effective amount of the antigen. There will be included an amount of antigen which will cause the subject to produce a specific and sufficient immunological response so as to impart protection to the subject from subsequent exposure to C. difficile. The antigen may be the disclosed non-toxic antigens. In one embodiment, the disclosed non-toxic antigens is administered by itself or in combination with an adjuvant.

Another aspect of the invention includes use of the disclosed non-toxic antigens as a subunit vaccine. A subunit vaccine refers to the use of a fragment of a pathogen as an inoculating agent. Those skilled in the art will know subunit vaccines offer a means to generate antibodies to a particular part or region of a pathogen.

Dosage schedule of administration and efficacy of the vaccine can be determined by methods known in the art. The amount of the vaccine and the immunization regimen may depend on the particular antigen and the adjuvant employed, the mode and frequency of administration, and the desired effect (e.g., protection and/or treatment). In general, the vaccine of the invention may be administered in amounts ranging between 1 μg and 100 mg, such as e.g. between 60 μg and 600 μg. A single dose of the vaccine comprising the disclosed non-toxic antigens may be in a range from about 1 μg to about 1 mg, preferably from about 5 μg to about 500 μg, more preferably from about 20 μg to about 200 μg.

The vaccine comprising the disclosed non-toxic antigens can be administered intrarectally, orally, intravenously, subcutaneously, intra-arterially, intramuscularly, intracardially, intraspinally, intrathoracically, intraperitoneally, intraventricularly, and/or sublingually.

The immunization regimen can be determined by methods known in the art. Administration of the vaccine can be repeated as is determined to be necessary by one skilled in the art. For example, a priming dose may be followed by 1, 2, 3 or more booster doses at weekly, bi-weekly or monthly intervals. In an embodiment of the present invention, the priming dose is followed by one or two booster administration in intervals from about 7 to about 14 days such as e.g. after 7 days and 21 days after first prime. In a preferred embodiment, the therapeutically effective amount of the vaccine is administered two or three times in intervals of 14 days+/−1, 2 or 3 days (bi-weekly) to a subject. In an embodiment of the present invention, the therapeutically effective amount of the vaccine is administered once.

Still another aspect is directed to the population which can be treated according to the present invention. In one embodiment, the population includes healthy individuals who are at risk of exposure to C. difficile, especially, the individuals impending hospitalization or residence in a care facility, as well as personals in hospitals, nursing homes and other care facilities. In another embodiment, the population includes previously infected patients who relapsed after discontinuation of antibiotic treatment, or patients for whom antibiotic treatment is not efficient.

In one more embodiment of the invention, the population includes individuals who are at least 18 years or more of age. In one preferred embodiment, the human subject is from 18 to 65 years old. In another preferred embodiment, the human subject is elderly individuals over 65 years of age. The latter age group being the most vulnerable population suffering from C. difficile infections. In some more embodiment, the human subject is younger than 18 years of age.

### Methods of Using the Disclosed Non-Toxic Antigens

The present invention also provides methods of using the disclosed non-toxic antigens. For example, the disclosed non-toxic antigens may be used to prevent or treat diseases associated with C. difficile. By way of example, introducing the isolated polypeptides of the present invention into the immune system of a subject may induce an immune response that includes the subject producing antibodies directed against the isolated polypeptide. Such antibodies are useful for recognizing C. difficile.

The present invention provides methods of delivering isolated polypeptides to a subject comprising administering the isolated polypeptide to a subject. The isolated polypeptide may be administered as a liquid or as a solid. The isolated polypeptide may further include a pharmaceutically acceptable carrier.

The present invention also provides methods for identifying and isolating variable domains of an antibody that recognize and bind to toxin A and or toxin B comprising use of the disclosed non-toxic antigens to produce an immune response, purifying and then characterizing the antibodies produced in response to the disclosed non-toxic antigens. Identified epitopes may be of use for cloning further antibodies or fragments thereof.

One aspect of the present invention is directed in part to the treatment, the prevention, and the detection of C. difficile. In some embodiments, a subject, such as an animal, receives treatment and/or prevention and/or detection of C. difficile. In other embodiments, the animal is a human. For example, the polypeptides of the present invention may be used to raise antibodies to C. difficile in vivo. By way of further example, the polypeptides of the present invention may be used to determine if a subject produces antibodies to C. difficile. In some embodiments, the polypeptide is used to isolate antibodies. By way of example, polypeptides may be bound to an affinity matrix.

By way of further example, the nucleic acid of the present invention can be used to transform and/transfect cells to recombinantly produce the polypeptides and/or antibodies of the present invention. The nucleic acids of the present invention may also be used, for example, to determine if a subject is infected with C. difficile. By way of example, this can be achieved using methods of radiolabeled hybridization.

By way of further example, the antibodies of the present invention can be used to recognize an infection by C. difficile. By way of example, the antibodies can recognize native toxin A and/or toxin B as an antigen. The antibodies of the present invention can also be used to fight an infection by C. difficile. By way of example, humanized antibodies or antibody fragments or monoclonal antibodies can employ a subject's own immune response to a C. difficile infection. By way of further example, the antibodies of the present invention may be coupled to a cytokine or a toxin or an enzyme or a marker to assist in treating and detecting an infection.

Further aspects of the present invention relate to diagnostic assays. The present invention is of use with many assays known in the art. Those skilled in the art will recognize the wide array of research based uses for the polypeptides, nucleic acids and antibodies of the present invention. The polypeptides, antibodies and nucleic acids of the present invention may, for example, be labeled, such as with a radioactive, chemiluminescent, fluorescent and/or dye molecules. The antibodies, nucleic acids and polypeptides of the present invention lend themselves to use assays for example DNA assays (such as southern blotting), RNA assays (such as northern blotting), protein assays (such as western blotting), chromatographic assays (such as gas, liquid, HPLC, size-exclusion), immunoassays (such as ELISA) and structural assays (such as crystallography and NMR spectroscopy). The antibodies, polypeptides and nucleic acids of the present invention may further be used as probes. Assays which amplify the signals from a probe are also known to those skilled in the art.

### Kits

The present invention provides kits comprising by way of example, and not limitation, nucleic acids encoding the disclosed non-toxic antigens. The kits may include one or more containers and instructions for use in accordance with any of the methods of the invention described herein. The disclosed non-toxic antigens may be used in a variety of assays including immunoassays for detecting C. difficile. In one embodiment, the disclosed non-toxic antigens serves to function as an antigen for the purposes of detecting antibody in biological samples. The containers may be unit doses, bulk packages (e.g., multi-dose packages) or sub-unit doses. The kits of this invention are in suitable packaging. Also contemplated are packages for use in combination with a specific device, such as an inhaler, nasal administration device or an infusion device. A kit may have a sterile access port. The container may also have a sterile access port. Kits may optionally provide additional components such as buffers and interpretive information.

The kits may be used to detect the presence of C. difficile or to detect a disease associated with C. difficile, such as CDAD. The kits may be used to prevent or treat diseases associated with C. difficile. The kits of the present invention may also be used to alleviate the symptoms of a disease associated with C. difficile.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Structural and Immunological Analysis of FIgG, a Clostridioides difficile Surface Antigen

Expression and Purification of 16 C. difficile surface Antigen candidates.

TABLE I
Twelve of the sixteen putative C. difficile surface antigens have been purified.

| Protein Description | MW (kDa) | Expressed? | Soluble or Insoluble? | Purified? |
|---|---|---|---|---|
| Polysaccharide deacetylase | 29.80 | Y | Sol. | Y |
| Glucosaminidase domian-cont. protein | 34.16 | N | — | N |
| N-acetylmuramoyl-L-alanine amidase | 36.01 | N | — | N |
| C40 peptidase 1 | 41.22 | Y | Insol. | Y |
| C40 peptidase 2 | 35.75 | Y | Insol. | Y |
| FlgK (flagellar hook-associated protein) | 50.16 | Y | Sol. | Y |
| FlgD (flagellar basal body mod. protein) | 24.14 | Y | Sol. | Y |
| FlgG (flagellar basal body rod) | 30.37 | Y | Sol. | Y |
| FlgE (flagellar hook-basal body protein) | 36.83 | Y | Sol. | Y |
| DUF3794 domina-containing protein | 21.08 | Y | Sol. | Y |
| S8 peptidase | 63.44 | N | — | N |
| N-acetylglucosaminidase | 65.60 | N | — | N |
| FliD (flagellar cap protein) | 58.53 | Y | Sol. | Y |
| NlpC/P60-family protein | 59.20 | Y | Insol. | Y |
| Cell wall protein 12 | 56.94 | Y | Insol. | Y |
| Cell wall protein 21 | 60.73 | Y | Insol. | Y |

—, no expression.

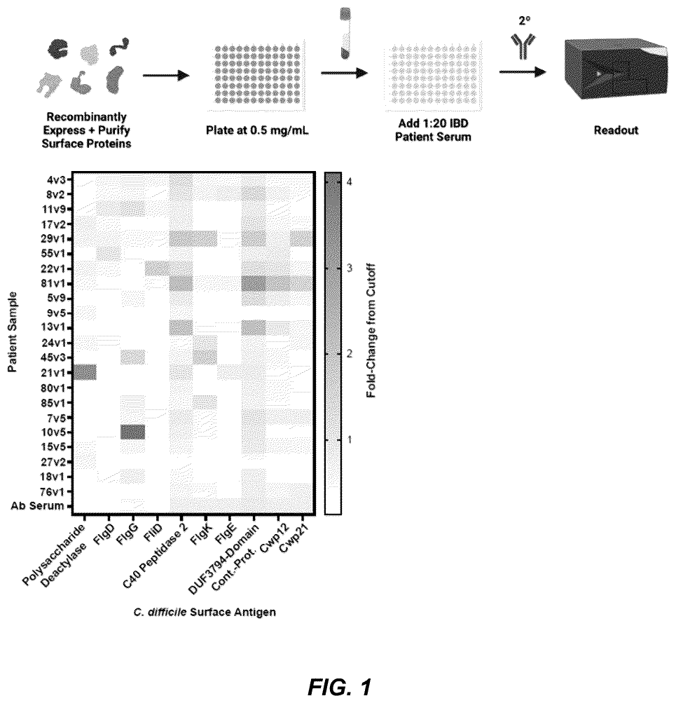

FIG. 1 shows testing for patient IgG reactivity against 10 C. difficile surface antigen candidates. Schematic of protocol testing CDI patient sera for IgGs against recombinantly-purified C. difficile surface antigen candidates (above). 1:20 IBD patient serum tested against candidate antigens at 1:500 IgG HRP conjugate. Mean values compared to experimental-defined cutoff, (3× serum control background); positive samples above one (below; n=22, performed in duplicate); Cwp=cell wall protein.

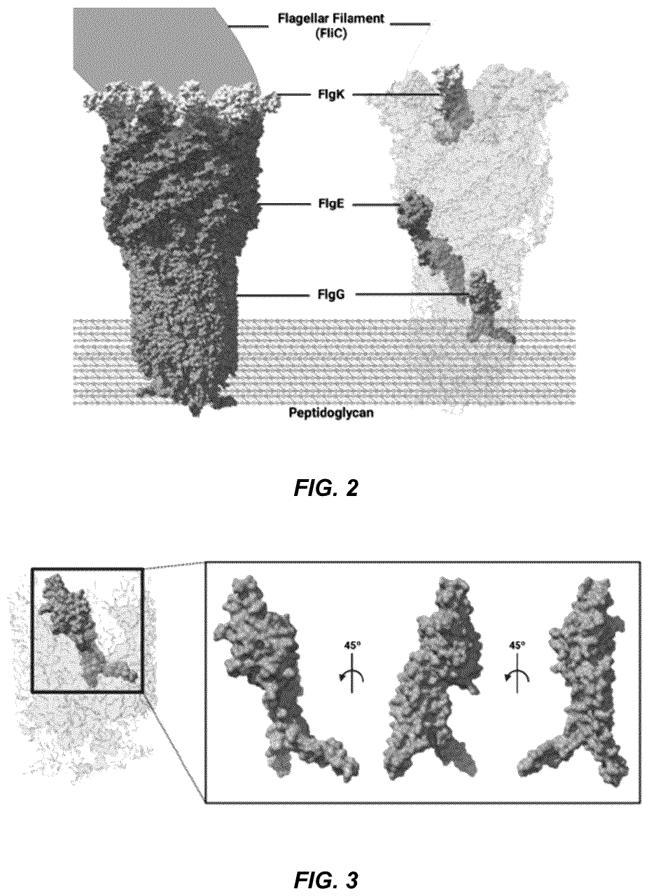

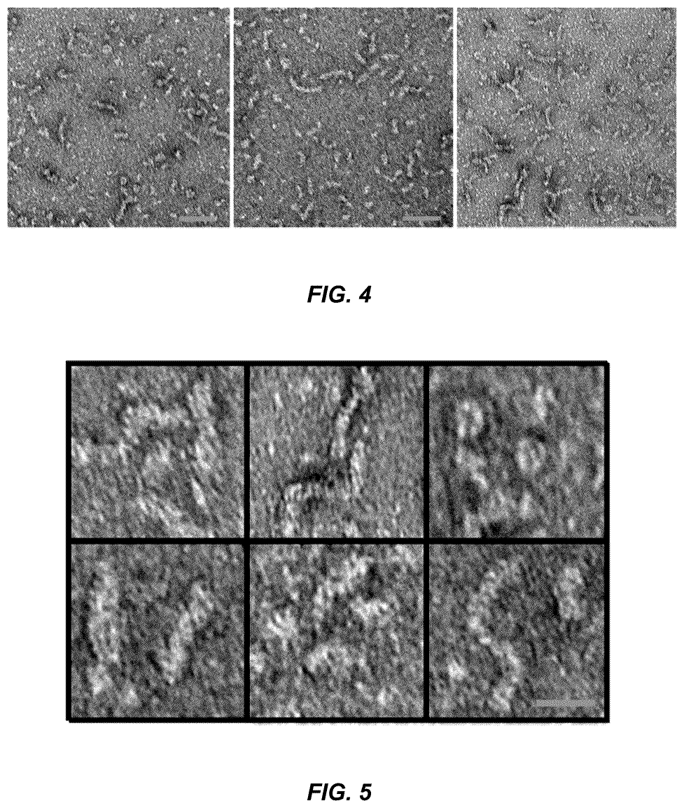

FIGS. 2 and 3 show structural characterization of FIgG. FIG. 2 shows a representation of a complete C. difficile flagellar basal body rod and hook based off Campylobacter jejuni FIgG and FIgE (PDB: 7CGO) and FIgK (PDB: 5XBJ), which share 29%, 36%, and 28% homology, respectively. Sequence homology is low due to differences in Gram negative and positive organisms, but structural homology is predicted to be retained. FIG. 3 shows FIgG is predicted to form a concentric, multi-layered tube of alpha-helical coiledcoils. Model based off C. jejuni, with 29% sequence homology (PDB: 7CGO).

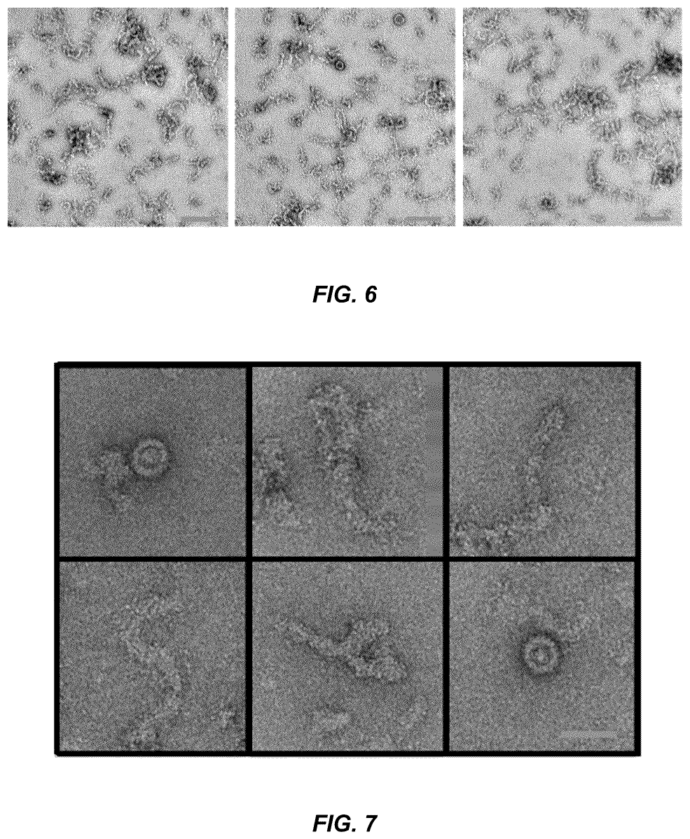

FIG. 4 shows negative-stained TEM images of FIgG at 28,000×. Oligomers appear to be 500-600 Å in length and concatenating (scale bar 1000 Å).

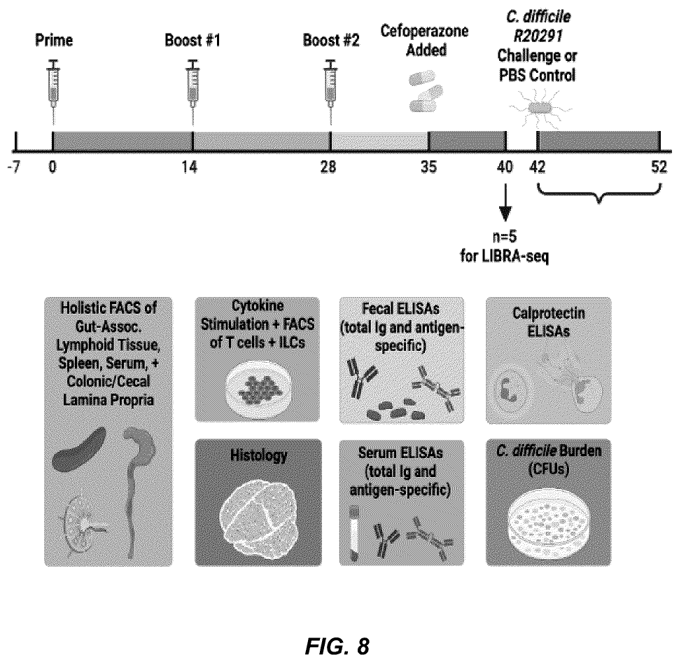

FIG. 5 shows negative-stained TEM images of oligomeric FIgG at 28,000×. Oligomeric FIgG appears to be concatenating in many views. Other views showcase potential top-down views of the FIgG concentric helix of coiled-coils (scale bar 300 Å).

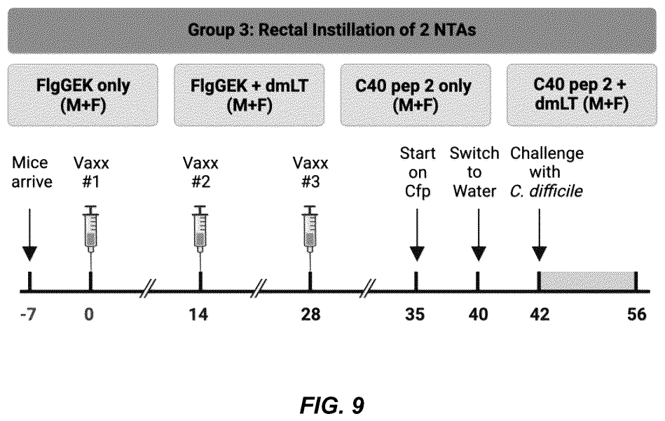

FIG. 6 shows negative-stained TEM images of FIgG+E+K complex at 28,000×. Concatenated complexes appear to be ˜800 Å in length, while singular complexes appear to be ˜350 Å (scale bar 1000 Å).

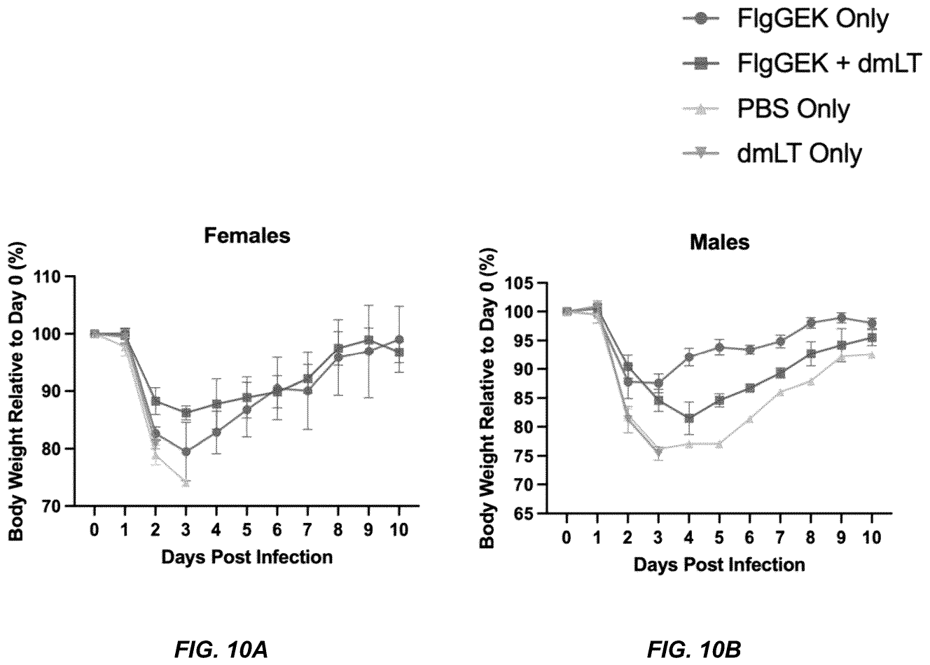

FIG. 7 contains negative-stained TEM images of the FIgG+E+K complex at 66,000×. Complexes appear to be concatenating in many views. Other micrographs showcase top-down views of the putative FIgE+K pore (scale bar 300 Å).

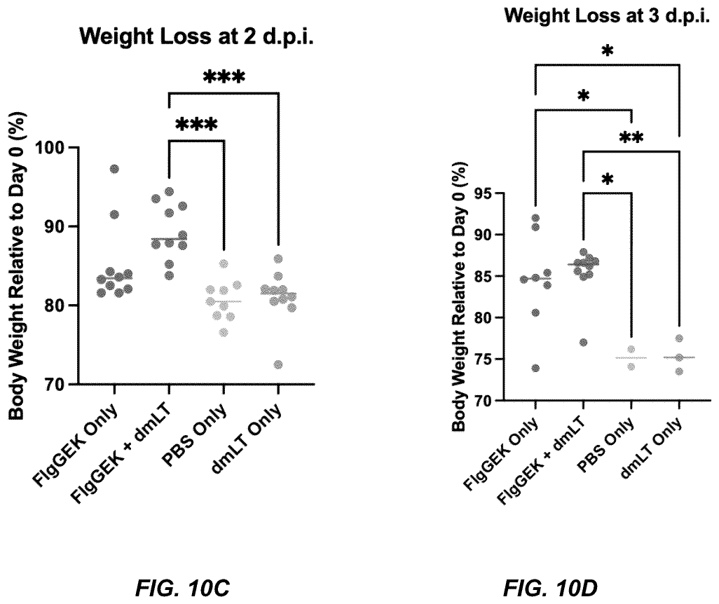

FIG. 8 shows vaccination timeline shown in days. B cells from 5 vaccinated mice from each group will be harvested for LIBRA-seq (linking B cell receptor to antigens specificity through sequencing) and the remaining mice will be challenged with C. difficile R20291 or PBS control, with desired metrics listed below. Cfp, cefoperazone.

### Example 2: Evaluate the Protective Capabilities of Novel C. difficile Non-Toxin Antigens In Vivo

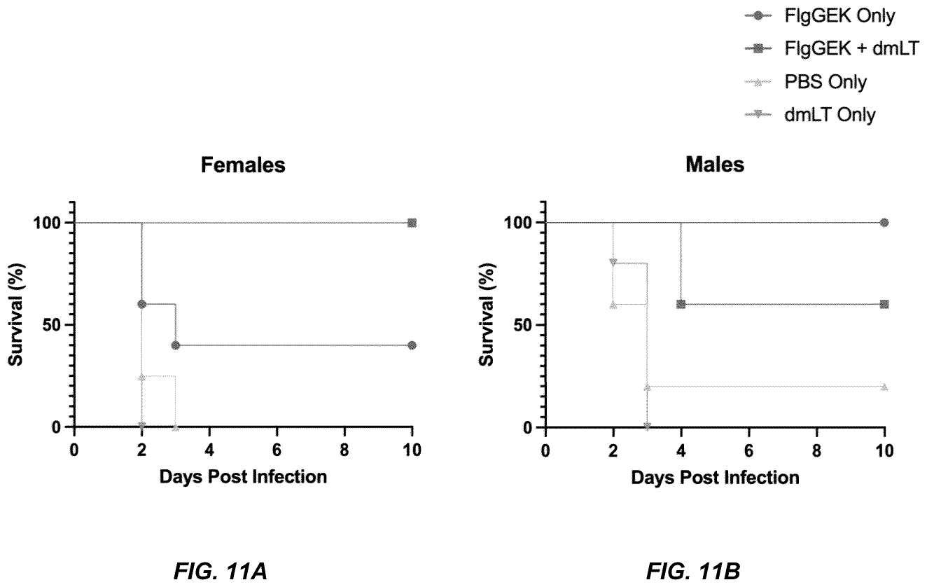

FIG. 9 is a schematic of vaccination scheme to determine non-toxin antigen(s) protect against morbidity and moribundity, reduce colonization, and elicit an anti-NTA humoral response. Group 3 involves rectal instillation of FIgGEK+C40 peptidase 2 (C40 pep 2); +/−dmLT. Group 4 involves rectal instillation of polysaccharide deacetylase+dmLT only.

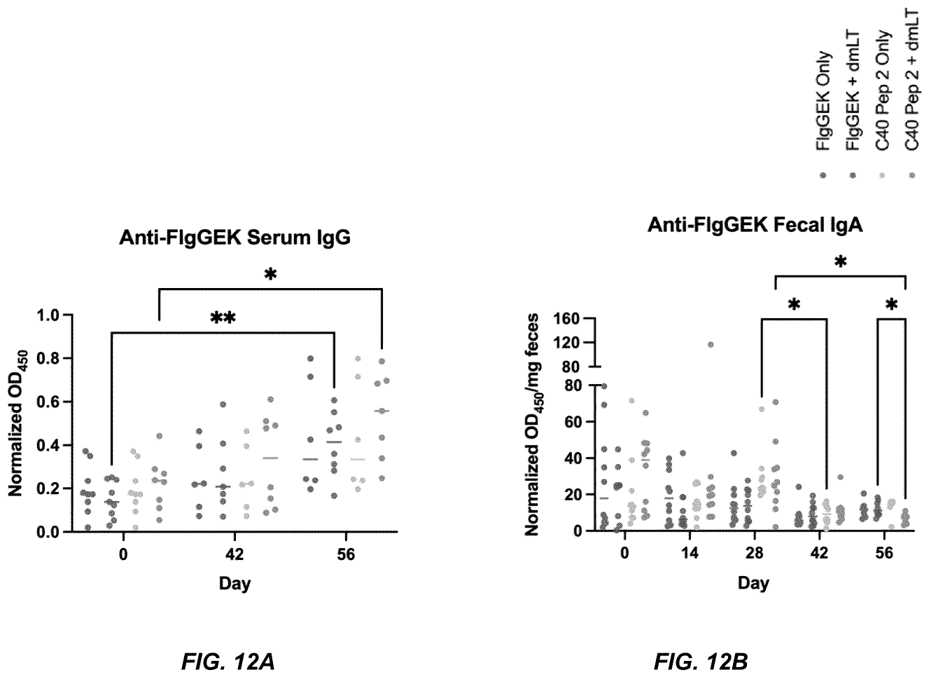

FIGS. 10A to 10D show rectal instillation of FIgGEK reduces weight loss in both sexes.

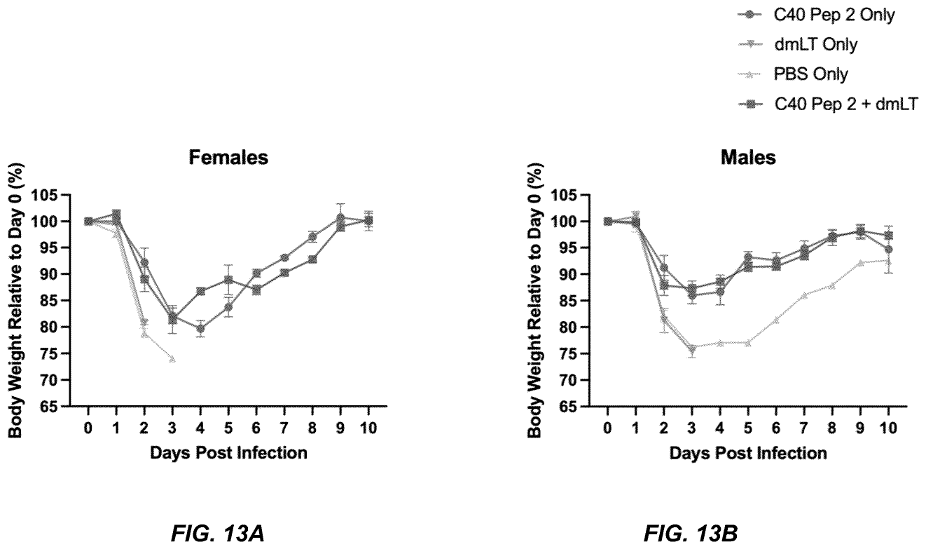

FIGS. 11A and 11B show rectal instillation of FIgGEK reduces death in both sexes.

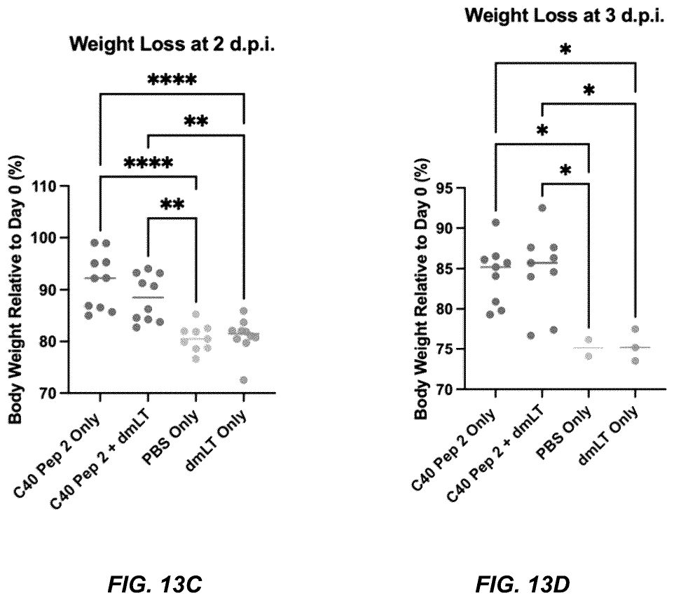

FIGS. 12A and 12B show rectal instillation of FIgGEK does not increase anti-FIgGEK serum IgG nor fecal IgA.

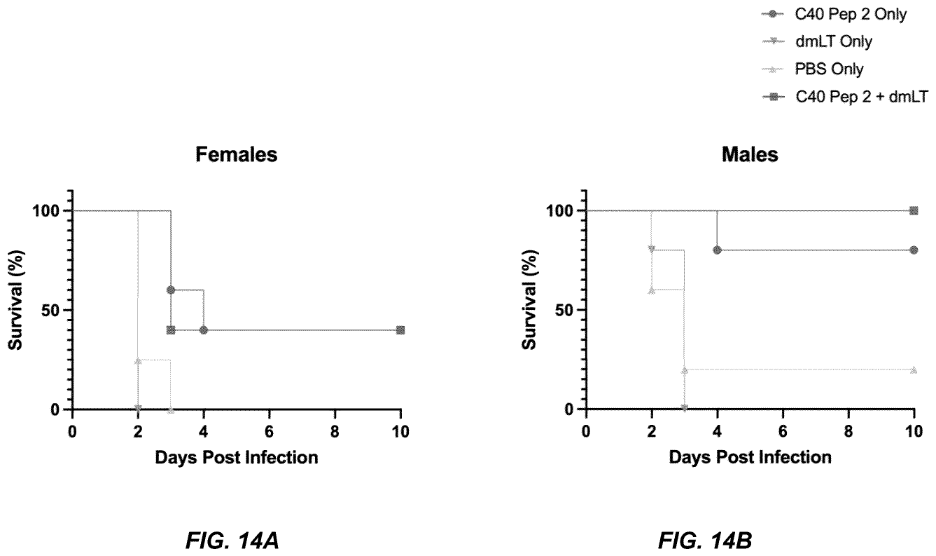

FIGS. 13A to 13D show rectal instillation of C40 pep 2 reduces weight loss in both sexes.

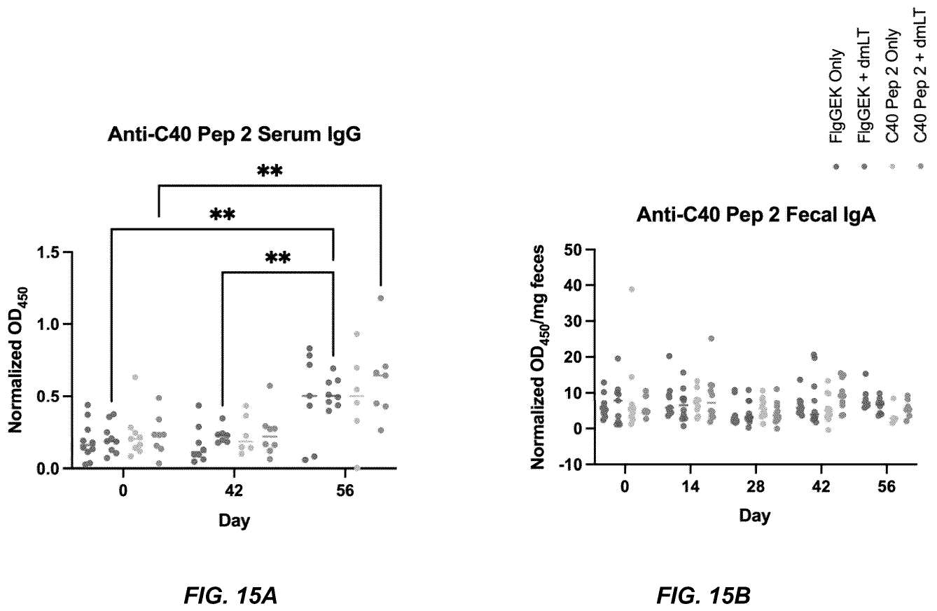

FIGS. 14A and 14B show rectal instillation of C40 pep 2 reduces death in both sexes.

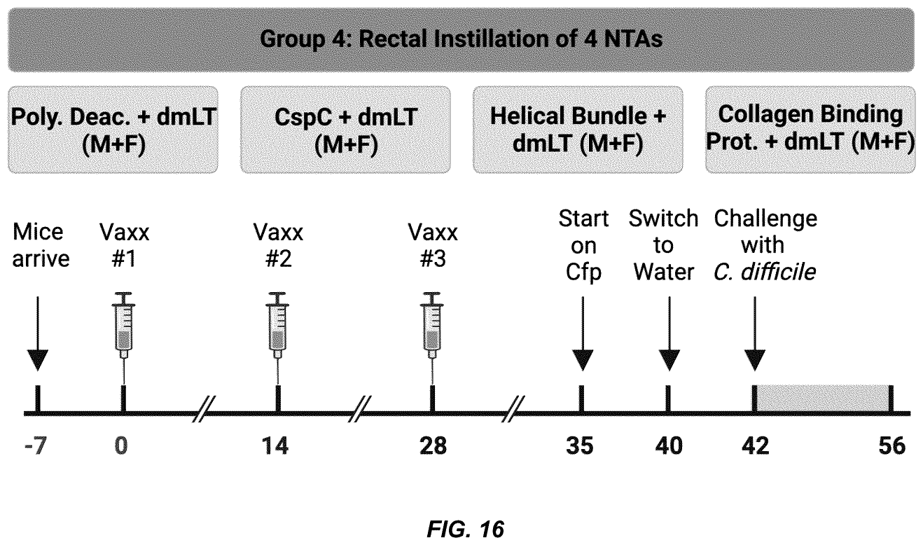

FIGS. 15A and 15B show rectal instillation of C40 pep 2 does not increase anti-C40 pep 2 serum IgG nor fecal IgA.

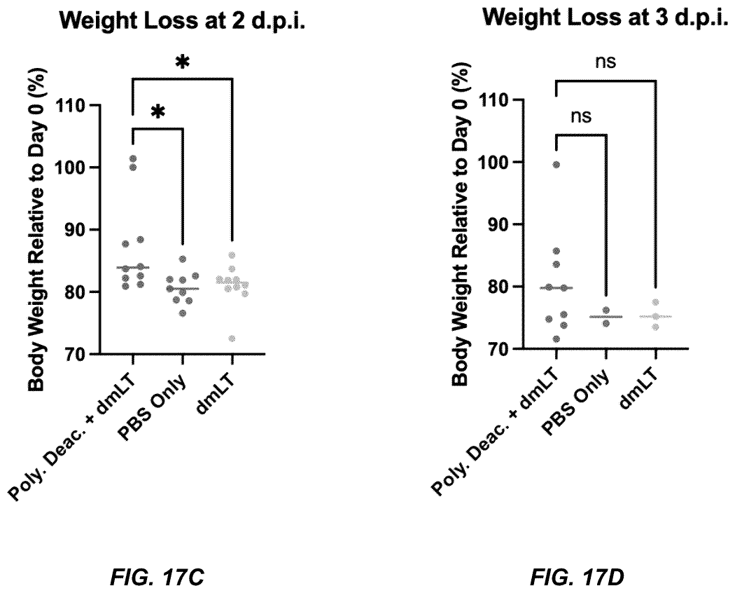

FIGS. 17A to 17D show rectal instillation of polysaccharide deacetylase reduces weight loss at 2 d.p.i.

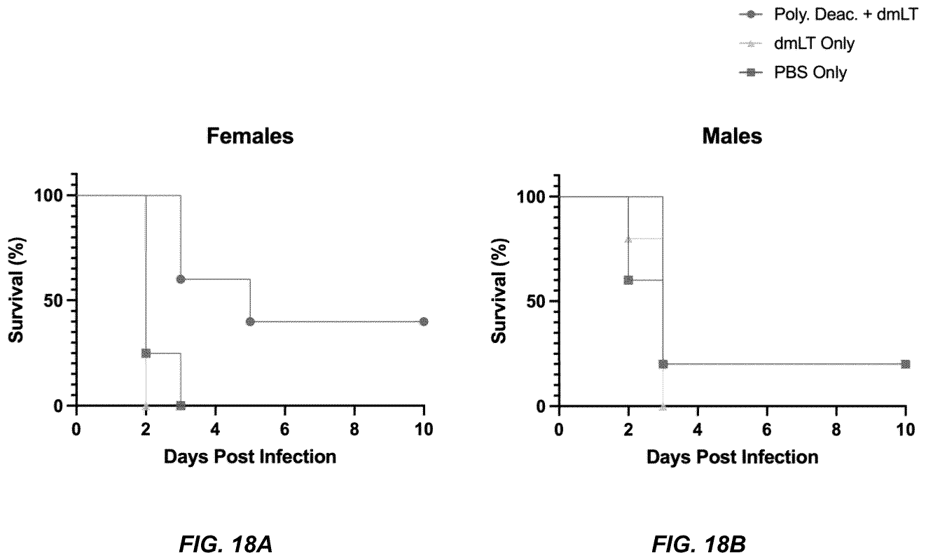

FIGS. 18A and 18B show rectal instillation of polysaccharide deacetylase prevents some death.

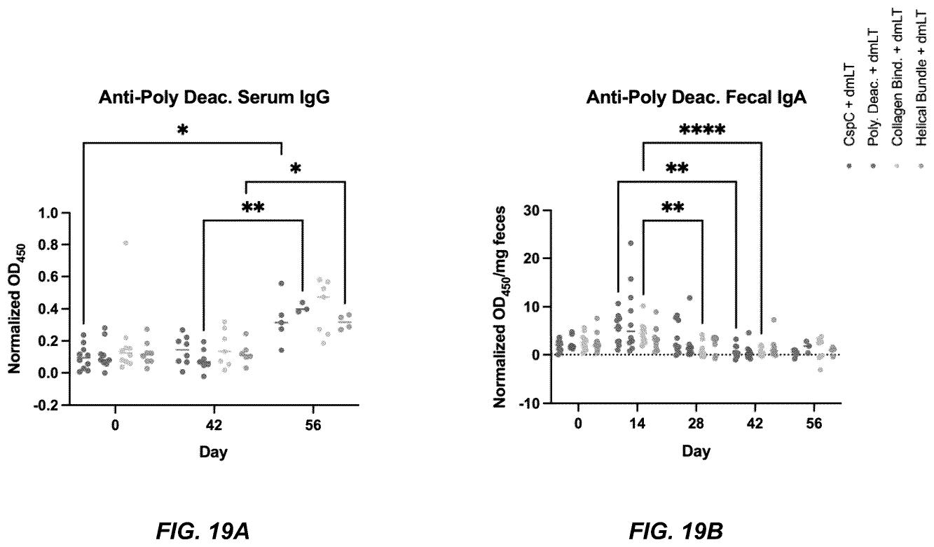

FIGS. 19A and 19B show rectal instillation of polysaccharide deacetylase does not increase anti-polysaccharide deacetylase serum IgG nor fecal IgA.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A vaccine composition comprising 1, 2, 3, 4, 5, 6, 7, 8, 9, 10, 11, 12, 13, 14, 15, or 16 C. difficile proteins (or immunogenic fragments thereof) selected from the group consisting of polysaccharide deacetylase, glucosaminidase domain-cont. protein, N-acetylmuramoyl-L-alanine amidase, C40 peptidase 1, C40 peptidase 2, FIgK (flagellar hook-associated protein), FlgD (flagellar basal body mod. protein), FIgG (flagellar basal body rod), FIgE (flagellar hook-basal body protein), DUF3794 domain-containing protein, S8 peptidase, N-acetylglucosaminidase, FliD (flagellar cap protein), NlpC/P60-family protein, Cell wall protein 12, and Cell wall protein 21, in a pharmaceutically acceptable carrier or excipient.

2. The vaccine composition of claim 1, comprising a combination of FIgG, FIgE, and FIgK proteins in a ternary complex.

3. The vaccine composition of claim 1, further comprising recombinantly-produced C. difficile toxins TcdA, TcdB, and/or CDT.

4. The vaccine composition of claim 1, further comprising an adjuvant.

5. The vaccine composition of claim 1, further comprising toxin antigens and an adjuvant.

6. The vaccine of claim 1, wherein the C. difficile proteins (or immunogenic fragments thereof) are in a cell-free preparation.

7. The vaccine of claim 1, wherein the C. difficile proteins (or immunogenic fragments thereof) are recombinant polypeptides.

8. A method for preventing, treating, or suppressing Clostridioides difficile infection in a subject, said method comprising administering to the subject a therapeutically effective amount of the vaccine composition of claim 1.

9. The method of claim 8, wherein the vaccine composition is administered intrarectally, orally or by enema, intravenously, subcutaneously, intramuscularly or transdermally.

10. The method of claim 8, further comprising administering to the subject a second agent.

11. The method of claim 10, wherein the second agent is an antibiotic.

12. The method of claim 11, wherein the antibiotic is vancomycin, metronidazole, or fidaxomicin.
